(19) United States
(12) Patent Application Publication
(10) Pub. No.: US 2012/0150300 A1
(43) Pub. Date: Jun. 14, 2012

(54) INTER-VERTEBRAL-BODY SPACER

(76) Inventor: Raj Nihalani, Irvine, CA (US)

(21) Appl. No.: 12/963,325

(22) Filed: Dec. 8, 2010

(51) Int. Cl. A61F 2/44

(52) U.S. Cl. 623/17.16

(57) ABSTRACT

An inter-vertebral-body (IVB) spacer may include a skeletal body and a pair of interchangeable contact pads. The skeletal body may be made of a rigid material for providing structure support, and the pair of contact pads may be made of a softer material for contacting the endplates of the vertebral bodies. Advantageously, the IVB spacer may be strong enough to withstand the pressure applied between the vertebral bodies yet soft enough to protect the vertebral bodies from being eroded. Moreover, the IVB spacer may have an inter-pad angle, which may be adjusted to adapt to patients with various vertebral bone structures. The inter-pad angle of the IVB spacer may be adjusted by simply selecting and replacing one of the contact pads.

### BACKGROUND

1. Field

The present invention relates generally to the field of medical devices used in spinal fusion surgery, and more particularly to inter-vertebral-body spacers.

2. Description of the Related Art

Spinal fusion (a.k.a. spondylodesis or spndylosyndesis) is a surgical technique used for joining two or more vertebrae. Spinal fusion surgery may be appropriate for patients who have neurological deficits or severe pain which has not responded to conservative treatment. For example, spinal fusion may be appropriate for patients who suffer from degenerative disc disease, spinal disc herniation, discogenic pain, spinal tumor, vertebral fracture, scoliosis, kyphosis, spondylolisthesis, spondylosis, posterior Rami syndrome, and/or other degenerative spinal conditions.

There are two main types of spinal fusion—posterolateral fusion and interbody fusion. In posterolateral fusion, a bone graft may be placed between the transverse processes of two successive vertebrae of a patient. In interbody fusion, a bone graft may be placed between two successive vertebral bodies of a patient. The surgeon may remove the spinal disc positioned between the two vertebral bodies, and may then place a spacer between the two vertebral bodies to maintain spine alignment and disc height. After that, the surgeon may fill the spacer with the bone graft, which may promote the fusion between the endplates of the two vertebral bodies.

Conventional spacers are generally made of one type of material, which may be either too hard for the endplates of the vertebral bodies or too brittle to withstand the pressure applied by the vertebral bodies. For example, a spacer made solely of rigid metallic material may be too hard for the endplates, and it may erode the endplates. For another example, a spacer made solely of plastic material may be too brittle to withstand the pressure applied by the vertebral bodies, and it may shatter shortly after installation. In either situation, the conventional spacers may cause internal injuries to the patient's body, and they may require frequent replacement.

Moreover, some conventional spacers are not adjustable. If a conventional spacer does not fit the vertebral body structure of a particular patient, a surgeon will need to replace that spacer and try a new one. According to standard surgical procedure, the spacer should be discarded once it is unpacked and has made contact with the patient. Hence, a good number of conventional spacers may be discarded during the fitting process. Because conventional spacers are generally expensive to manufacture, the practice of discarding unused spacers may drive up the cost of spinal fusion surgeries.

Thus, there is a need to provide inter-vertebral-body spacer with improved durability and adjustability.

### SUMMARY

The present invention may provide an inter-vertebral-body (IVB) spacer for use in spinal fusion surgery. The IVB spacer may be made of a rigid material for providing structural support and a softer material for contacting the endplates of the vertebral bodies. Advantageously, the IVB spacer may be strong enough to withstand the pressure applied between the vertebral bodies yet soft enough to protect the vertebral bodies from being eroded. Moreover, the IVB spacer may have an inter-pad angle, which may be adjusted to adapt to patients with various vertebral bone structures. The inter-pad angle of the IVB spacer may be adjusted by selecting and replacing one of the contact pads. As such, only a small portion of the IVB spacer will be discarded during the adjustment process while the majority portion of the IVB spacer will be fully utilized. Advantageously, the IVB spacer may help reduce overall equipment costs of the spinal fusion surgery and provide a better and more accurate and comfortable fit to the patient.

In one embodiment, an inter-vertebral-body (IVB) spacer may be used for placement between first and second vertebral bodies, and the spacer may comprise a first pad having a first contact surface configured to contact the first vertebral body, a second pad having a second contact surface configured to contact the second vertebral body, and a skeletal body positioned between the first and second pads, and having a first frame configured to secure the first pad, support the first contact surface, and arrange the first contact surface along a first plane, a second frame configured to secure the second pad, support the second contact surface, and arrange the second contact surface along a second plane, such that the second plane forms an inter-pad angle with the first plane, and a plurality of pillars coupled between the first and second frames, and configured to maintain a distance between the first and second frames.

In another embodiment, an inter-vertebral-body (IVB) spacer may be used for placement between first and second vertebral bodies, and the spacer may comprise a first thermal plastic pad having a first contact surface configured to contact the first vertebral body, a second thermal plastic pad having a second contact surface configured to contact the second vertebral body, a rigid skeletal body positioned between the first and second pads, and having a first frame configured to secure the first pad, support the first contact surface, and arrange the first contact surface along a first plane, a second frame configured to secure the second pad, support the second contact surface, and arrange the second contact surface along a second plane, such that the second plane forms an inter-pad angle with the first plane, and a plurality of pillars coupled between the first and second frames, and configured to maintain a distance between the first and second frames, and a stopper configured to be coupled to the rigid skeletal body and prevent the first and second thermal plastic pads from detaching from the first and second frames.

In yet another embodiment, an inter-vertebral-body (IVB) spacer may be used for placement between first and second vertebral bodies, and the spacer may comprise a first thermal plastic pad having a first contact surface configured to contact the first vertebral body, a second thermal plastic pad having a second contact surface configured to contact the second vertebral body, a rigid skeletal body positioned between the first and second pads, and having a first frame configured to secure the first pad, support the first contact surface, and arrange the first contact surface along a first plane, a second frame configured to secure the second pad, support the second contact surface, and arrange the second contact surface along a second plane, such that the second plane forms an inter-pad angle with the first plane, and a plurality of pillars coupled between the first and second frames, and configured to maintain a distance between the first and second frames, a stopper configured to be coupled to the rigid skeletal body and prevent the first and second thermal plastic pads from detaching from the first and second frames, and an anchoring device disposed between the first and second frames of the rigid skeletal body, and configured to protrude the first and second thermal plastic pads and anchor the rigid skeletal body to the first and second vertebral bones.

### BRIEF DESCRIPTION OF THE DRAWINGS

Other systems, methods, features, and advantages of the present invention will be or will become apparent to one with skill in the art upon examination of the following figures and detailed description. It is intended that all such additional systems, methods, features, and advantages be included within this description, be within the scope of the present invention, and be protected by the accompanying claims. Component parts shown in the drawings are not necessarily to scale, and may be exaggerated to better illustrate the important features of the present invention. In the drawings, like reference numerals designate like parts throughout the different views, wherein:

FIG. 1A shows a perspective view of an inter vertebral body (IVB) spacer being placed in between two vertebral bodies according to an embodiment of the present invention;

FIG. 1B shows a perspective view of the IVB spacer according to an embodiment of the present invention;

FIG. 1C shows a top view of the IVB spacer being placed on top of a vertebral body according to an embodiment of the present invention;

FIGS. 2A-2E show various perspective views of the IVB spacer including the assembly process of the IVB spacer according to an embodiment of the present invention;

FIGS. 3A-3C show various dimensions of the IVB spacer according to an embodiment of the present invention;

FIGS. 4-6 show the side views of the IVB spacers with various inter-pad angles according to various embodiments of the present invention;

FIGS. 7A-7C show a perspective view, an exploded view, and a side view of an IVB spacer having an anchoring device according to an embodiment of the present invention;

FIGS. 8A-8B show the deployment of the anchoring device according to an embodiment of the present invention;

FIGS. 9A-9B show a perspective view and an exploded view of an IVB spacer according to an alternative embodiment of the present invention; and

FIGS. 10A-10D show various views of an alternative skeletal body according to an embodiment of the present invention.

### DETAILED DESCRIPTION

Apparatus, systems and methods that implement the embodiment of the various features of the present invention will now be described with reference to the drawings. The drawings and the associated descriptions are provided to illustrate some embodiments of the present invention and not to limit the scope of the present invention. Throughout the drawings, reference numbers are re-used to indicate correspondence between reference elements. In addition, the first digit of each reference number indicates the figure in which the element first appears.

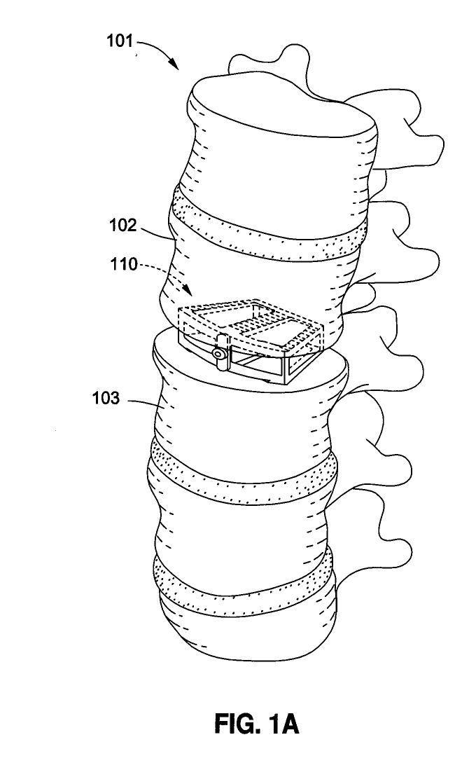

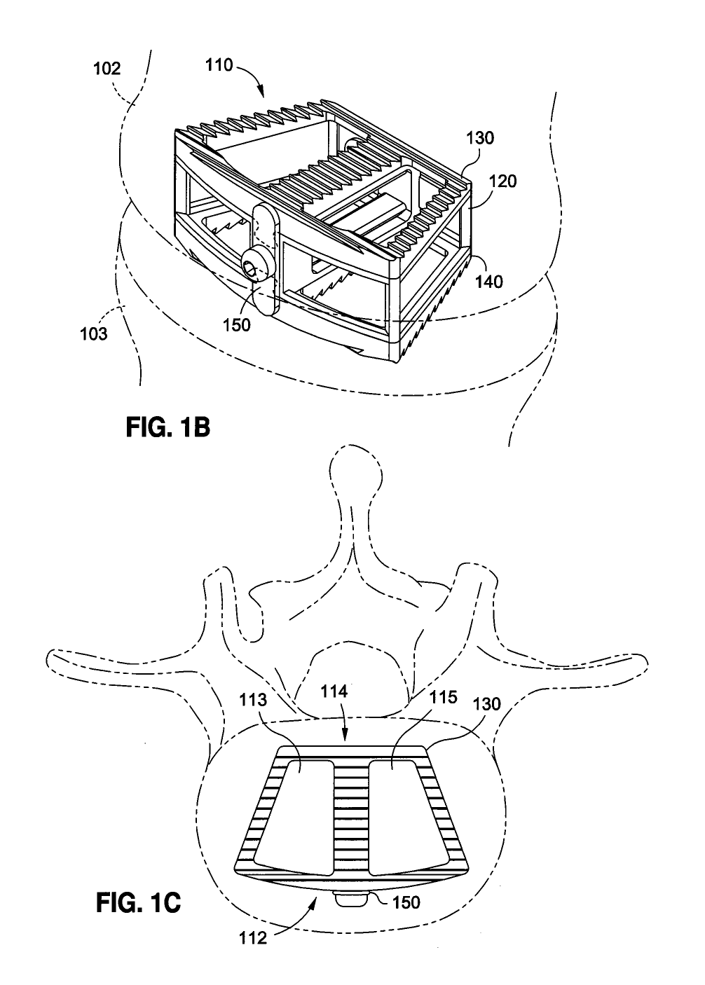

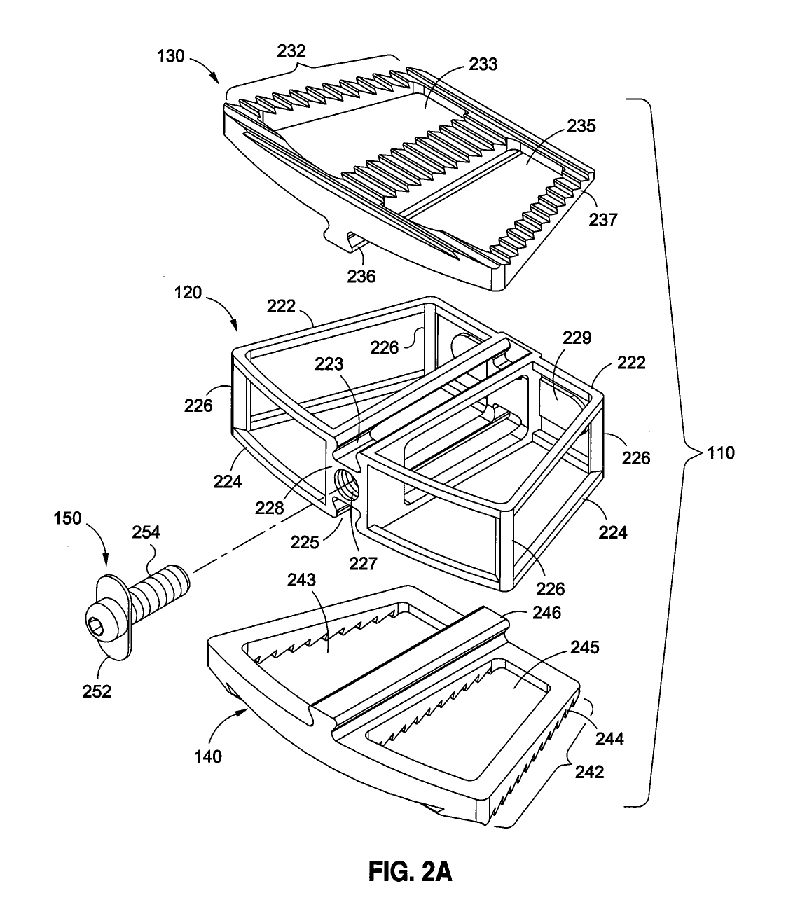

FIG. 1A shows a perspective view of an inter-vertebral-body (IVB) spacer 110 being placed in between two vertebral bodies 102 and 103 according to an embodiment of the present invention. Generally, the IVB spacer 110 may be used in inter-vertebral-body (interbody) fusion surgery for facilitating the fusion of two or more vertebral bodies. A patient may have a defective spinal column 101, which may be caused by a damaged or degenerated inter-vertebral disc between a first vertebral body 102 and a second vertebral body 103. To correct the defective spinal column 101, a surgeon may remove the damaged or degenerated inter-vertebral disc and then insert the IVB spacer 110 in between the first and second vertebral bodies 102 and 103.

FIG. 1B shows a perspective view of the IVB spacer 110 according to an embodiment of the present invention. Generally, the IVB spacer 110 may include a first (top) pad, a second (bottom) pad 140, a skeletal body (case) 120, and a stopper 150. The first pad 130 may be used for contacting the first vertebral body 102, while the second pad 140 may be used for contacting the second vertebral body 103. The skeletal body 120 may be used for supporting the first and second pads 102 and 103. The stopper 150 may be used for preventing the first and second pads 130 and 140 from being detached from the skeletal body 120. Together, the skeletal body 120 and the first and second pads 130 and 140 may provide structural support and stabilization between the first and second vertebral bodies 102 and 103. The skeletal body 120 may be made of non-brittle and/or rigid materials. The first and second pads 130 and 140 may be made of high endurance and corrosive resistance materials. Moreover, the first and second pads 130 and 140 may be made of a material having a modulus that is approximately the same as the modulus of the human bone.

In one embodiment, for example, the skeletal body 120 may be made of metal, metal alloy, ceramic, pyrolictic carbon, and/or carbon fiber, while the first and second pads 130 and 140 may be made of ceramic, pyrolictic carbon, carbon fiber, and/or thermal plastic material. In another embodiment, for example, the skeletal body 120 may be made of titanium, titanium alloys, titanium oxide, cobalt chrome, 316L stainless steel, nitinol and/or nickel alloys, while the first and second pads 130 and 140 may be made of (polyether ether ketone) PEEK material and/or ultra high molecular weight polyethylene (UHMWPE). In yet another embodiment, for example, the skeletal body 120 may be made of titanium, while the first and second pads 130 and 140 may be made of PEEK material.

FIG. 1C shows a top view of the IVB spacer 110 being placed on top of the second vertebral body 103. The IVB spacer 110 may have a front side 112 and a back side 114. Particularly, the front side 112 may have a wide curvy profile aligning with the anterior ridge side of the vertebral body 103, while the narrower back side 114 may have a linear profile aligning with the posterior ridge side of the second spinal segment 103. The IVB spacer 110 may have one or more fusion channels, which may allow a bone putty material (autologous bone graft) to fill up a space between the first and second vertebral bodies 102 and 103. For example, the IVB spacer 110 may have a first (left) fusion channel 113 and a second (right) fusion channel 115, both of which may extend vertically through the IVB spacer 110. Accordingly, the bone putty material may contact and connect the first and second vertebral bodies 102 and 103 by filling up the first and second fusion channels 113 and 115.

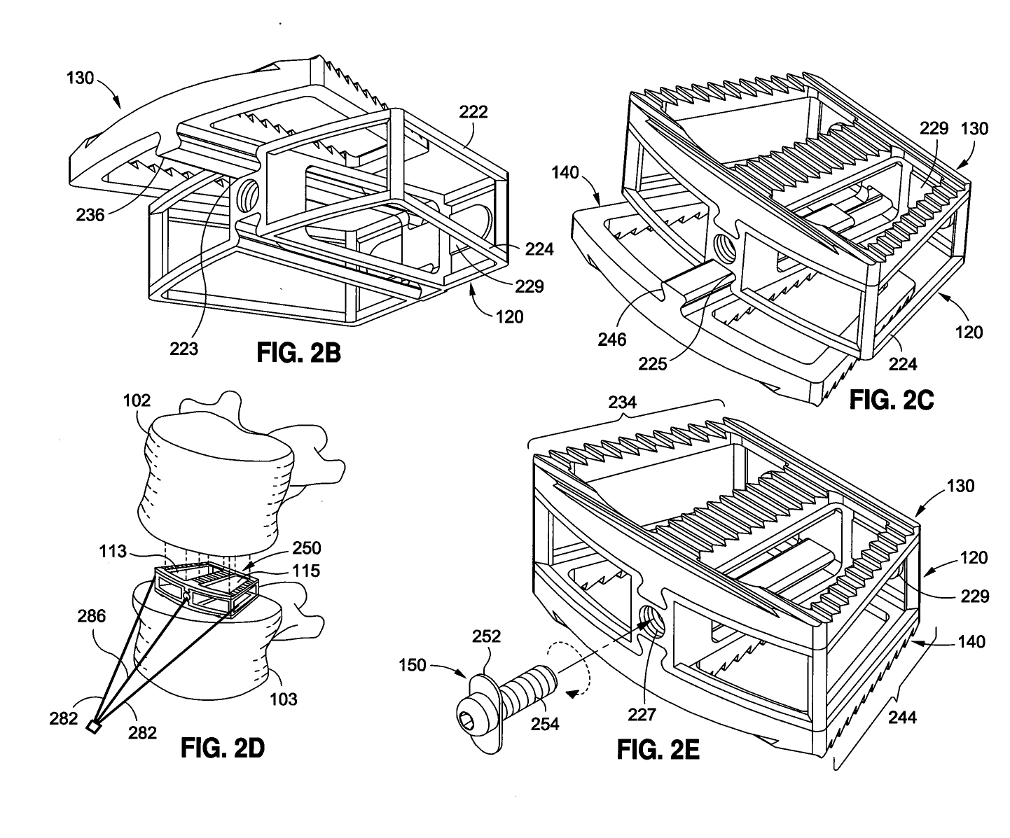

FIG. 2A shows an exploded view of the IVB spacer 110. Generally, the first and second pads 130 and 140 may share similar structural features. For example, the first pad 130 may have a first contact surface 232 for contacting the first vertebral body 102, and the second pad 140 may have a second contact surface 242 for contacting the second vertebral body 103. For another example, the first pad 130 may have a first assembly surface 237 for contacting one side of the skeletal body 120, and the second pad 140 may have a second assembly surface 247 for contacting another side of the skeletal body 120. The first contact surface 232 and the first assembly surface 237 may form a first insertion angle, while the second contact surface 242 and the second assembly surface 247 may form a second insertion angle. Together, the first and second insertion angles may be combined to form an inter-pad angle, which may exactly match or be substantially similar to a lordotic angle between the first and second vertebral bodies 102 and 103 of a particular patient.

Moreover, the first pad 130 may have first and second fusion openings 233 and 235, and the second pad 140 may have third and fourth fusion openings 243 and 245. Together, the first and third fusion openings 233 and 243 may help define the first fusion channel 113, while the second and fourth fusion openings 235 and 245 may help define the second fusion channel 115.

Furthermore, the first and second pads 130 and 140 may include one or more alignment devices and/or mechanisms, which may be used for aligning the first and second pads 130 and 140 with the skeletal body 120. For example, the first pad 130 may have a first alignment bar 236, and the second pad 140 may have a second alignment bar 246. In one embodiment, each of the first and second alignment bars 236 and 246 may be implemented by a rectangular bar with a rectangular cross section. In another embodiment, each of the first and second alignment bars 236 and 246 may be implemented by a rectangular bar with a willow-tail cross section.

The skeletal body 120 may be used for supporting and securing the first and second pads 130 and 140. Generally, the skeletal body 120 may include a first (top) frame 222, a second (bottom) frame 224, and a plurality of pillars 226, which may be coupled between the first and second frames 222 and 224. The first frame 222 may be used for contacting and supporting the first assembly surface 237 of the first pad 130, while the second frame 224 may be used for contracting and supporting the second assembly surface 247 of the second pad 140. The plurality of pillars 226 may be used for providing structural support between the first and second frames 222 and 224, such that the first and second frames 222 and 224 may maintain a constant and evenly distributed distance.

To align the first pad 130 with the skeletal body 120, the first frame 222 may have a first alignment trench for receiving and guiding the first alignment bar 236 of the first pad 130. As shown in FIG. 2B, the first alignment bar 236 may be inserted into the first alignment trench 223. The first pad 130 may slide from the front side 112 to the back side 114 of the first frame 222. Because of the willow-tail cross section, the first alignment trench 223 may prevent the first alignment bar 236 from moving in a direction that is perpendicular to the first frame 222. That is, the first alignment trench 223 may allow the first alignment bar 236 to move only in a parallel direction with respect to the first frame 222.

To align the second pad 140 with the skeletal body 120, the second frame 224 may have a second alignment trench for receiving and guiding the second alignment bar 246 of the second pad 140. As shown in FIG. 2C, the second alignment bar 246 may be inserted into the second alignment trench 225. The second pad 140 may slide from the front side 112 to the back side 114 of the second frame 224. Because of the willow-tail cross section, the second alignment trench 225 may prevent the second alignment bar 246 from moving in a direction that is perpendicular to the second frame 224. That is, the second alignment trench 225 may allow the second alignment bar 246 to move only in a parallel direction with respect to the second frame 224.

The first and second pads 130 and 140 may be replaced and exchanged easily. As such, surgeons may optimize the result of interbody fusion procedures by trying and switching one of the first and second pads 130 and 140. Particularly, surgeons may select the first or second pad 130 or 140 with the appropriate geometric properties and material characteristic. For example, surgeons may select the first and second pads 130 and 140 from a group of pads that have a specific range of insertion angles. For another example, surgeons may select the first and second pads 130 and 140 from a group of pads that have a specific shape and size. For yet another example, surgeons may select the first and second pads 130 and 140 from a group of pads that are made of a specific type material. As a result, surgeons may keep at least one of the two pads and the skeletal body 120 during the optimization process. Advantageously, less material may go into waste during the optimization process, and the equipment cost of the interbody fusion surgery may therefore be reduced. This process can be repeated after the interbody fusion surgery due to change in condition of the patient. Particularly, one of the first and second pads 130 and 140 may be removed and replaced by another pad, which may have similar or different geometric properties and material characteristics.

After the first and second pads 130 and 140 are properly received, aligned, and secured by the respective first and second frames 222 and 224 of the skeletal body 120, a partially assembled IVB spacer 250 may be formed. As shown in FIG. 2D, the partially assembled IVB spacer 250 may be handled by a pair of surgical forceps 282. In one embodiment, the skeletal body 120 may include a pair of manipulation trails to provide easy access for the pair of surgical forceps 282. An insertion driver 286 may be used for inserting the partially assembled IVB spacer 250 into the space between the first and second vertebral bodies 102 and 103. To cooperate with the insertion driver 286, the skeletal body 120 may have a center member 228 coupled between the first and second frames 222 and 224. The center member 228 may define an installation channel 227, which may be used for receiving the insertion driver 286.

After the partially assembled IVB spacer 250 is properly placed between the first and second vertebral bodies 102 and 103, the stopper 150 may be used for locking the first and second pads 130 and 140 in place. Generally, the stopper 150 may include a screw head 256, a threaded shaft 254, and a flange 252 coupled between the flange 252 and the screw head 256. The threaded shaft 254 may be inserted into the installation channel 227 and engage an inner threaded section (not shown) therein. The screw head 256 may receive a locking force, thereby causing the threaded shaft 254 to further engage the installation channel 117. When the threaded shaft 254 substantially engages the installation channel 117, the screw head 256 may cause the flange 252 to push against the front sides of the first and second pads 130 and 140. The flange 252 may block the first and second pads 130 and 140, such that the first and second alignment bars 236 and 246 are stopped from sliding out of the first and second alignment trenches 223 and 225. When the IVB spacer 110 is fully assembled, the flange 252 may prevent the first and second pads 130 and 140 from being detached from the first and second frames 222 and 224.

The first and second pads 130 and 140 may include one or more anchoring devices or mechanisms to restrain the movement of the IVB spacer 110 with respect to the first and second vertebral bodies 102 and 103. In one embodiment, for example, the first contact surface 232 of the first pad 130 may have a first set of spikes (keels) 234, and the second contact surface 242 of the first pad 140 may have a second set of spikes (keels) 244. The first and second set of spikes (keels) 234 and 244 may incline or point away from the direction of insertion.

Once the IVB spacer 110 is properly positioned between the first and second vertebral bodies 102 and 103, the first set of spikes 234 may anchor the first pad 130 to the endplate of the first vertebral body 102, while the second set of spikes 244 may anchor the second pad 140 to the endplate of the second vertebral body 103. Accordingly, the first and second sets of spikes 234 and 244 may help stabilize the IVB spacer 110 between the first and second vertebral bodies 102 and 103. Moreover, the first and second sets of spikes 234 and 244 may help align the first and second vertebral bodies 102 and 103 and hold them in place. Advantageously, the IVB spacer 110 may promote the stability between the first and second vertebral bodies 102 and 103.

After the IVB spacer 110 is properly positioned and anchored between the first and second vertebral bodies 102 and 103, a surgeon may inject the bone putty into the IVB spacer 110 via the front openings of the skeletal body 120. The bone putty may reach and contact the first and second vertebral bodies 102 and 103 via the first and second fusion channels 113 and 115. The first fusion channel 113 may be a passage that may coincide with the first fusion opening 233 of the first pad 130, a left segment of the top frame 222, a left segment of the second frame 224, and the third fusion opening 243 of the second pad 140. Similarly, the second fusion channel 115 may be a passage that may coincide with the second fusion opening 235 of the first pad 130, a right segment of the top frame 222, a right segment of the second frame 224, and the fourth fusion opening 245 of the second pad 140.

To prevent the bone putty from reaching the spinal cord, the skeletal body 120 may include a back stopper 229, which may be used for stopping the bone putty from leaving the back side of the skeletal body 120. After the bone putty substantially occupies the first and second fusion channels 113 and 114, the first vertebral body 102 may be connected to the second vertebral body 103 by the bone putty. As a result, the interbody fusion between the first and second vertebral bodies 102 and 103 may take place.

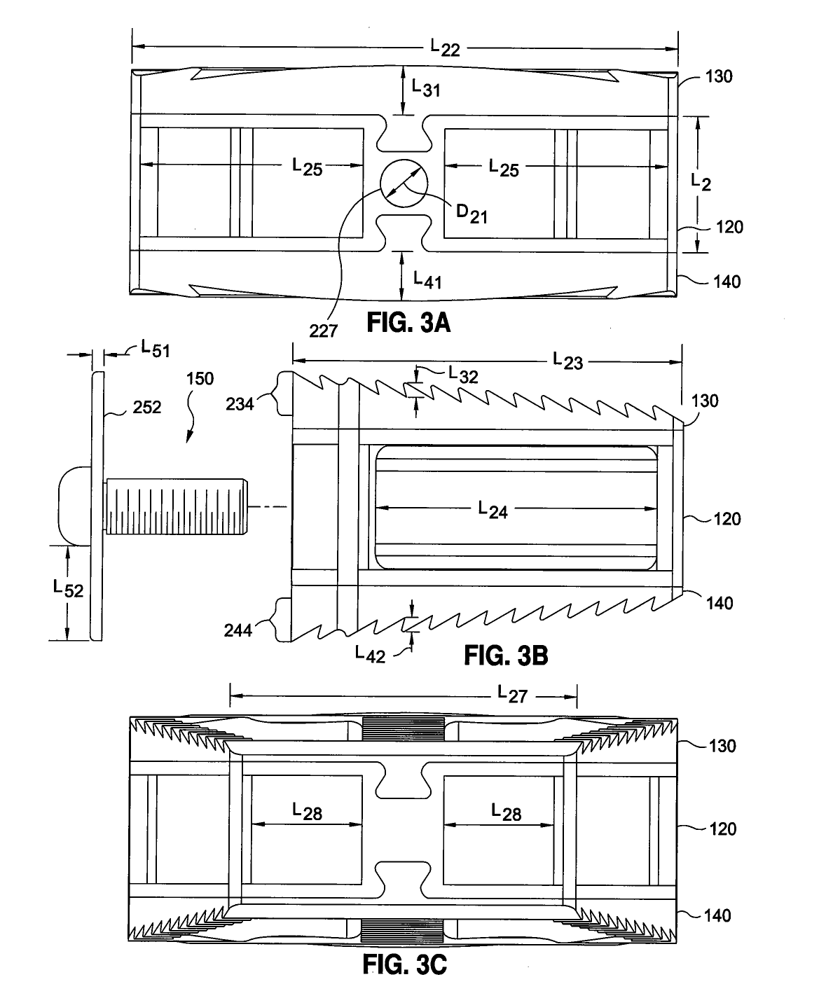

The discussion now turns to various dimensions of the IVB spacer 110. FIG. 3A-3C show various dimensions of the IVB spacer. Referring to FIG. 3A, the first pad 130 may have a first front thickness L₃₁, which may range, for example, from about 1.5 mm to about 5.5 mm, and the second pad 140 may have a second front thickness L₄₁, which may range, for example, from about 1.5 mm to about 5.5 mm. In one embodiment, the first front thickness L₃₁ may be about 3.5 mm, and the second front thickness L₄₁ may be about 3.5 mm.

The skeletal body 120 may have a skeletal height L₂₁, which may range from about 5 mm to about 15 mm, and a front skeletal width L₂₂, which may range from about 15 mm to about 40 mm. In one embodiment, the skeletal height L₂₁ may be about 10 mm, and the front skeletal width L₂₂ may be about 30 mm. The front openings of the skeletal body 120 may each have a front opening width L₂₅, which may range, for example, from about 12 mm to about 18 mm. In one embodiment, the front opening width L₂₅ may be about 15 mm. The installation channel 227 may have a diameter D₂₁, which may range, for example, from about 2.5 mm to about 4.5 mm. In one embodiment, the diameter D₂₁ may be about 3.5 mm.

Referring to FIG. 3B, the first set of spikes 234 may each have a first spike height L₃₂, which may range, for example, from about 0.5 mm to about 0.7 mm, and the second set of spikes 244 may each have a second spike height L₄₂, which may range, for example, from about 0.5 mm to about 0.7 mm. In one embodiment, the first and second spike height L₃₂ and L₄₂ may each be about 0.6 mm.

The flange 252 of the stopper 150 may have a flange thickness L₅₁, which may range, for example, from about 0.5 mm to about 1 mm, and a flange height L₅₂, which may range, for example, from about 5 mm to about 7 mm. In one embodiment, the flange thickness L₅₁ may be about 0.75 mm, and the flange height L₅₂ may be about 5.6 mm. The skeletal body 120 may have a skeletal body length L₂₃, which may range, for example, from about 20 mm to about 30 mm. In one embodiment, the skeletal body length L₂₃ may be about 25 mm. The side openings of the skeletal body 120 may have a side opening width L₂₄, which may range, for example, from about 15 mm to about 25 mm. In one embodiment, the side opening width L₂₄ may be about 25 mm.

Referring to FIG. 3C, the skeletal body 120 may have a back skeletal width L₂₇, which may range, for example, from about 20 mm to about 30 mm. In one embodiment, the back skeletal width L₂₇ may be about 23 mm. The back openings of the skeletal body 120 may each have a back opening width L₂₈, which may range, from about 7 mm to about 9 mm. In one embodiment, the back opening width L₂₈ may be about 8 mm.

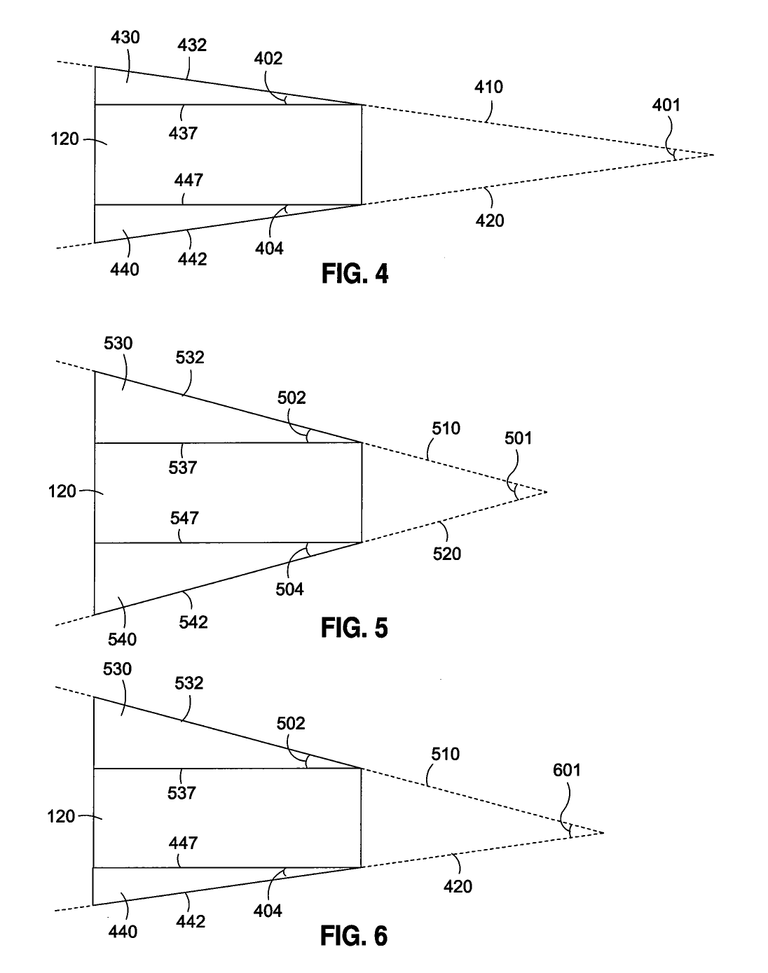

The discussion now turns to the adjustability of the inter-vertebral-body (IVB) spacer 110. FIGS. 4-6 show the side views of the IVB spacers 110 with various inter-pad angles according to various embodiments of the present invention. Referring to FIG. 4, the first pad 430 may have a first narrow insertion angle 402, which may be formed between the first contact surface 432 and the first assembly surface 437. The second pad 440 may have a second narrow insertion angle 404, which may be formed between the second contact surface 442 and the second assembly surface 447. The first and second narrow insertion angles 402 and 404 may each range, for example, from about 1 degree to about 7 degrees. In one embodiment, the first and second insertion angles 402 and 404 may each be about 3.5 degrees.

After being secured by the skeletal body 120, the first contact surface 432 of the first pad 130 may be arranged to align with a first plane 410, and the second contact surface 442 of the second pad 440 may be arranged to align with a second plane 420. Together, the first and second planes 410 and 420 may form a narrow inter-pad angle 401, which may range, for example, from about 4 degrees to about 10 degrees. In one embodiment, the narrow inter-pad angle 401 may be about 7 degrees. Because the first assembly surface 437 is arranged to be substantially parallel with the second assembly surface 447, the sum of the first and second narrow insertion angles 402 and 404 may determine the value of the narrow inter-pad angle 401. Hence, by selecting the first and second pads 430 and 440 with narrow insertion angles, the IVB spacer 110 may adapt to patients with small lordotic angles between successive vertebral bodies.

Referring to FIG. 5, the first pad 530 may have a first wide insertion angle 502, which may be formed between the first contact surface 532 and the first assembly surface 537. The second pad 540 may have a second wide insertion angle 504, which may be formed between the second contact surface 542 and the second assembly surface 547. The first and second wide insertion angles 502 and 504 may each range, for example, from about 5 degrees to about 10 degrees. In one embodiment, the first and second wide insertion angles 502 and 504 may each be about 7 degrees.

After being secured by the skeletal body 120, the first contact surface 532 of the first pad 530 may be arranged to align with a third plane 510, and the second contact surface 542 of the second pad 540 may be arranged to align with a fourth plane 520. Together, the third and fourth planes 510 and 520 may form a wide inter-pad angle 501, which may range, for example, from about 10 degrees to about 20 degrees. In one embodiment, the wide inter-pad angle 501 may be about 14 degrees. Because the first assembly surface 537 is arranged to be substantially parallel with the second assembly surface 547, the sum of the first and second wide insertion angles 502 and 504 may determine the value of the wide inter-pad angle 501. Hence, by selecting the first and second pads 530 and 540 with wide insertion angles, the IVB spacer 110 may adapt to patients with large lordotic angles between successive vertebral bodies.

Referring to FIG. 6, the first pad 530 may have the first wide insertion angle 502, which may be formed between the first contact surface 532 and the first assembly surface 537. The second pad 440 may have the second narrow insertion angle 404, which may be formed between the second contact surface 442 and the second assembly surface 447.

After being secured by the skeletal body 120, the first contact surface 532 of the first pad 530 may be arranged to align with the third plane 510, and the second contact surface 442 of the second pad 440 may be arranged to align with the second plane 420. Together, the third and second planes 510 and 420 may form an intermediate inter-pad angle 601, which may range, for example, from about 7 degrees to about 15 degrees. In one embodiment, the intermediate inter-pad angle 601 may be about 10 degrees. Because the first assembly surface 537 is arranged to be substantially parallel with the second assembly surface 447, the sum of the first wide and second narrow insertion angles 502 and 404 may determine the value of the intermediate inter-pad angle 601. Hence, by selecting the first and second pads 530 and 440, the IVB spacer 110 may adapt to patients with medium lordotic angles between successive vertebral bodies.

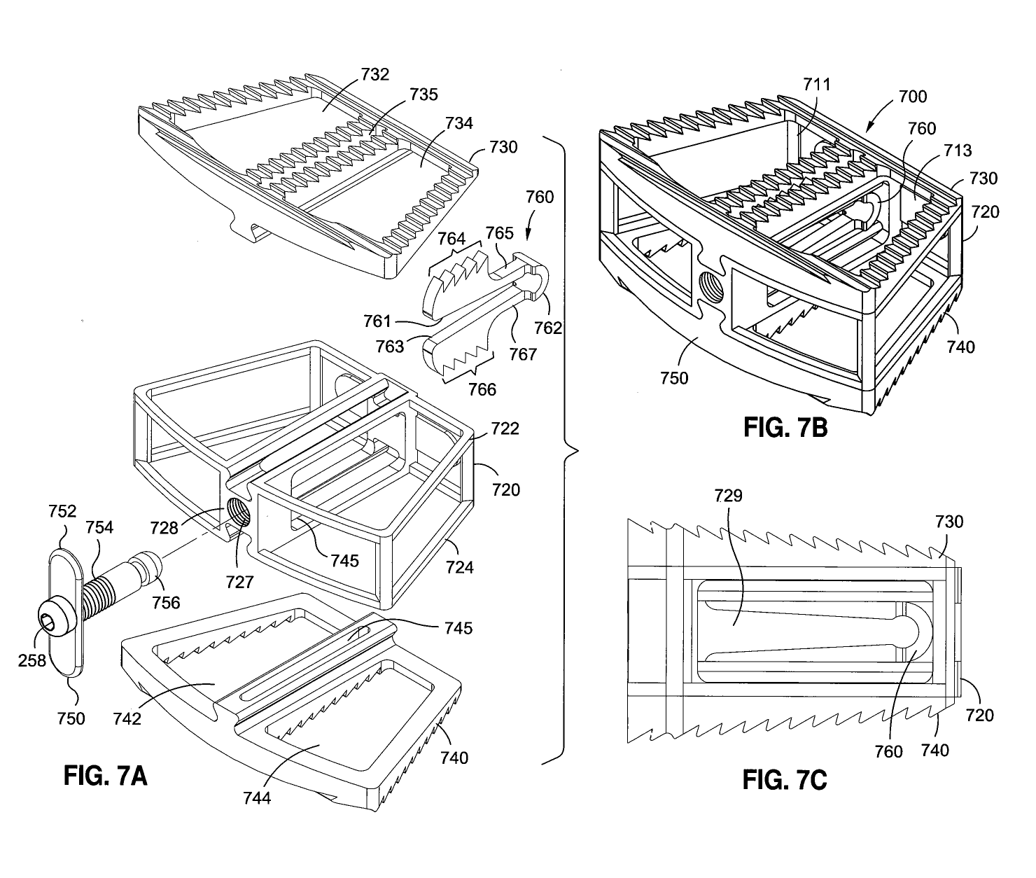

In FIGS. 7A-7C, an alternative inter-vertebral-body (IVB) spacer 700 is shown according to an alternative embodiment of the present invention. The alternative IVB spacer 700 may be similar to the IVB spacer 110. For example, the alternative IVB spacer 700 may include a skeletal body 720, which may have a first frame 722, a second frame 724, and a plurality of pillars 726 coupled between the first and second frames 722 and 724. For another example, the alternative IVB space 700 may include a first pad 730, which may be coupled to the first frame 722, and a second pad 740, which may be coupled to the second frame 724. The first and second pads 730 and 740 may be used for contacting the first and second vertebral bodies 102 and 103, respectively. The skeletal body 720 may be used for supporting the first and second pads 730 and 740.

The skeletal body 720 may be made of rigid materials, while the first and second pads 730 and 740 may be made of high endurance and corrosive resistance material. In one embodiment, for example, the skeletal body 720 may be made of metal, metal allowy, ceramic, pyrolictic carbon, and/or carbon fiber, while the first and second pads 730 and 740 may be made of ceramic, pyrolictic carbon, carbon fiber, and/or thermal plastic material. In another embodiment, for example, the skeletal body 720 may be made of titanium, titanium alloys, titanium oxide, cobalt chrome, 316L stainless steel, nitinol and/or nickel alloys, while the first and second pads 730 and 740 may be made of polyether ether ketone (PEEK) material and/or ultra high molecular weight polyethylene (UHMWPE). In yet another embodiment, the skeletal body 720 may be made of titanium, while the first and second pads 730 and 740 may be made of PEEK material.

Despite the above similarities, the alternative IVB spacer 700 may be different from the IVB spacer 110 in at least one aspect. For example, the IVB spacer 700 may include an anchoring device 760, which may be used for anchoring the IVB spacer 700 in between two successive vertebral bodies. The anchoring device 760 may be an anchoring clip, which may be positioned in the center of the skeletal body 720. The anchoring device 760 may be positioned between the first and second frames 722 and 724 of the skeletal body 720. More specifically, the anchoring device 760 may be disposed within a center compartment 729, which may be positioned adjacent to the first and second fusion channels 711 and 713. The anchoring device 760 may be held by the center compartment 729, such that the anchoring device 760 may be restrained from having any lateral or longitudinal movement in relative to the skeletal body 720.

The anchoring device 760 may include a first keel section 764, a second keel section 766, a first lever 765 coupled to the first keel section 764, a second level 767 coupled to the second keel section 766, and a flexible hinge 762 coupled between the first and second levers 765 and 767. The flexible hinge 762 may allow a relative movement between the first and second keel sections 764 and 766. More specifically, the first and second keel sections 764 and 766 may move away from each other when a pair of deployment forces is directed against the first and second inner surfaces 761 and 763. The first and second keel sections 764 and 766 may resume to their original positions when the pair of deployment forces is removed.

The keels of the first and second keel sections 764 and 766 may incline or point towards the direction of insertion of the IVB spacer 700. When deployed, the keels of the first and second keel sections 764 and 766 may engage the first and second vertebral bodies 102 and 103, respectively. As a result, the keels of the first and second keel sections 764 and 766 may anchor the IVB spacer 700 to the first and second vertebral bodies 102 and 103 and prevent the IVB spacer 700 from moving towards the posterior side of the first and second vertebral bodies 102 and 103.

After the first and second pads 730 and 740 are properly aligned, received, and secured by the skeletal body 720, the anchoring device 760 may be inserted into the skeletal body 720. For example, the anchoring device 760 may be inserted into the skeletal body 720 via the installation channel 727. Next, the stopper 750 may be coupled to the installation channel 727. The stopper 750 may have a screw head 758, a threaded section 754 coupled to the screw head 758, a flange 752 coupled between the threaded section 754 and the screw head 758, and a deployment head 756 coupled to the threaded section 754.

The screw head 758 may be used for receiving a locking force, which may cause the threaded section 754 to be substantially engaged to the installation channel 727. The flange 752 may be used for preventing the first and second pads 730 and 740 from being detached from the skeletal body 720. The deployment head 756 may be used for deploying the anchoring device 760. Advantageously, the stopper 750 may be used for simultaneously locking the first and second pads 730 and 740 and deploying the anchoring device 760.

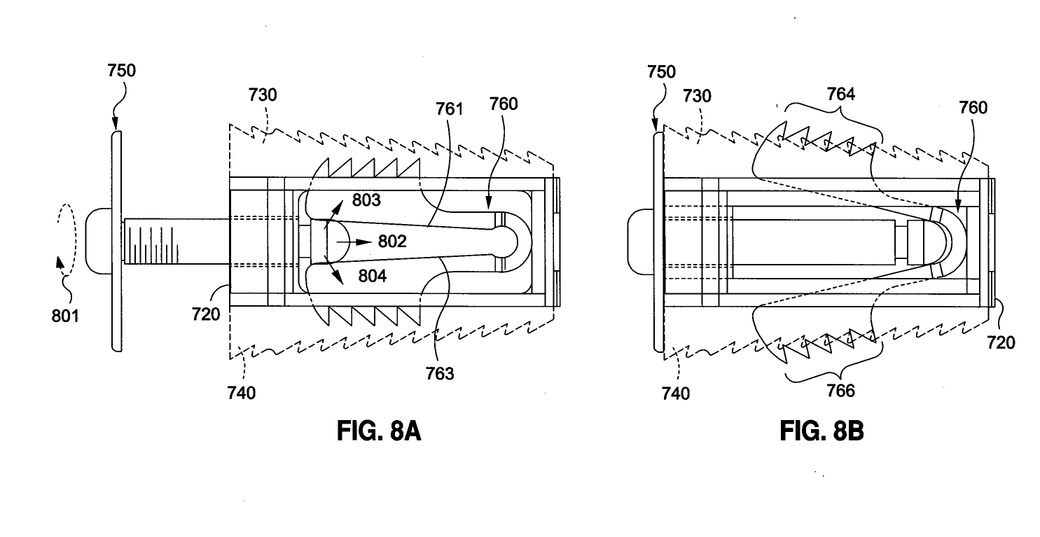

When the IVB spacer 700 is being properly positioned between the first and second vertebral bodies 102 and 103, the anchoring device 760 may be deployed. FIGS. 8A-8B show the process of deploying the anchoring device 760 according to an embodiment of the present invention. Initially, the deployment head 756 of the stopper 750 may contact the first and second inner surfaces 761 and 763 of the anchoring device 760. As the screw head 758 receives the locking force 801, the threaded section 754 may be substantially engaged to the installation channel 727.

The substantial engagement between the threaded section 754 and the installation channel 727 may create an insertion force 802, which may push the deployment head 756 against the first and second inner surfaces 761 and 763 of the anchoring device 760. As a result, the deployment head 756 may assert the pair of deployment forces 803 and 804 against the first and second inner surfaces 761 and 763, respectively, thereby pushing the first and second keel sections 764 and 766 away from each other.

FIG. 8B shows a side view of the IVB spacer 700 when the anchoring device 760 is deployed. At the deployed state, the first keel section 764 may protrude from the first frame 722 of the skeletal body 720 and then from a first slot 735 of the first pad 730. Similarly, the second keel section 766 may protrude from the second frame 724 of the skeletal body 720 and then from a second slot 745 of the second pad 740. The keels of the first and second keel sections 764 and 766 may incline or point towards the insertion direction of the IVB spacer 700.

Upon engaging the first and second vertebral bodies 102 and 103, the keels of the first and second keel sections 764 and 766 may prevent the IVB spacer 700 from moving towards the posterior side of the first and second vertebral bodies 102 and 103. The anchoring device 760 and the first and second pads 730 and 740 may cooperate with one another to substantially stabilize the IVB spacer 700 in between the space defined by the first and second vertebral bodies 102 and 103. The stability provided the IVB spacer 700 may advantageously enhance the quality and rate of interbody fusion.

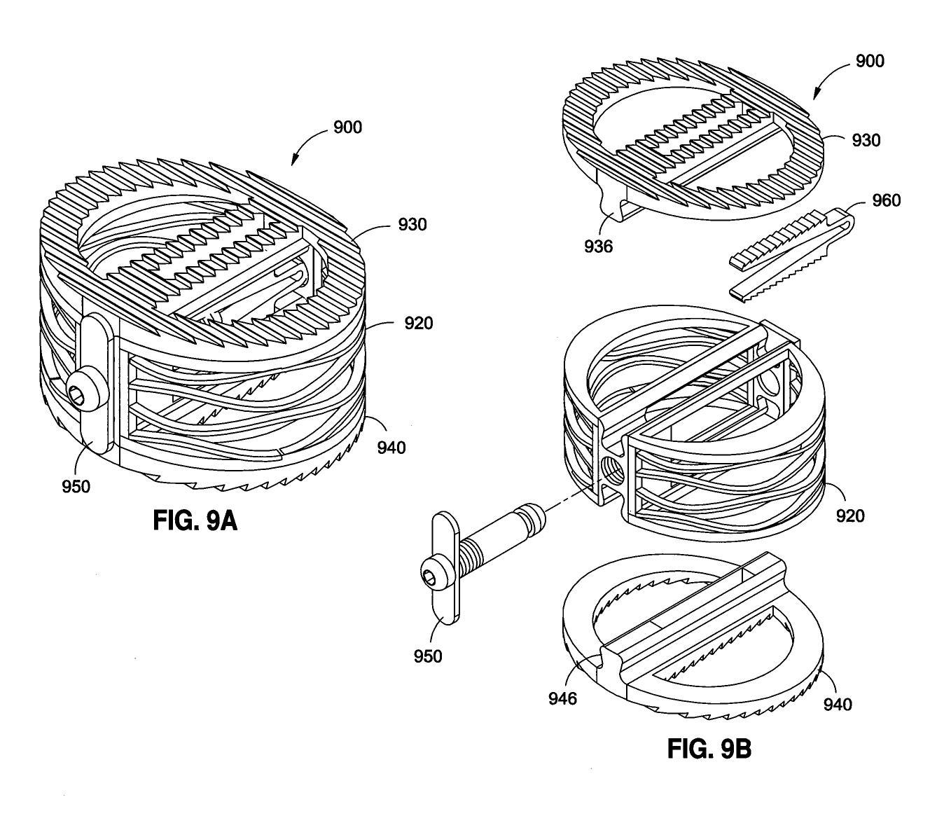

FIGS. 9A-9B show a perspective view and an exploded view of an IVB spacer 900 according to an alternative embodiment of the present invention. Generally, the IVB spacer 900 may include a first ellipsoidal pad 930, a second ellipsoidal pad 940, a skeletal body 920, a stopper 950, and an anchoring device 960. The skeletal body 920 may be coupled between the first and second ellipsoidal pads 930 and 940. The anchoring device 960 may be positioned within the skeletal body 920, and it may be deployed when the stopper 950 is substantially engaged to the skeletal body 920.

The first and second ellipsoidal pads 930 and 940 may be similar to the first and second pads 130 and 140 as discussed in FIGS. 2A-2E. For example, the first ellipsoidal pad 930 may have a first contact surface and a first assembly surface, and the second ellipsoidal pad 940 may have a second contact surface and a second assembly surface. The first and second contact surfaces may each have a set of spikes inclining or pointing away from the direction of insertion. As such, the set of spikes may be used for anchoring the respective first or second ellipsoidal pads 930 or 940 to the endplates of the vertebral bodies.

The first ellipsoidal pad 930 may have a first alignment bar 936 formed on the first assembly surface, and the second ellipsoidal pad 940 may have a second alignment bar 646 formed on the second assembly surface. The first and second alignment bars 936 and 946 may be received and secured by the skeletal body 920. As such, the first and second ellipsoidal pads 930 and 940 may be attached to the skeletal body 920. Moreover, the stopper 950 may be used for preventing the first and second ellipsoidal pads 930 and 940 from being detached from the skeletal body 920.

Before the stopper 950 is substantially coupled to the skeletal body 920, the first and second ellipsoidal pads 930 and 940 may be slid in and out of the skeletal body 920 relatively easily. The first and second ellipsoidal pads 930 and 940 may come with different insertion angles and/or sizes to accommodate patients with various vertebral body structures. A surgeon may adjust the IVB spacer 900 by simply switching one of the first and second ellipsoidal pads 930 and 940. That is, the surgeon does not need to discard the whole IVB spacer 900 during the adjustment process.

Advantageously, most parts of the IVB spacer 900 may be fully utilized, and the equipment costs of the spinal fusion may be reduced. Moreover, the first and second ellipsoidal pads 930 and 940 may each have a shape that matches the footprint of the endplates of the vertebral bodies. Therefore, the first and second ellipsoidal pads 930 and 940 may provide good contact with the vertebral bodies.

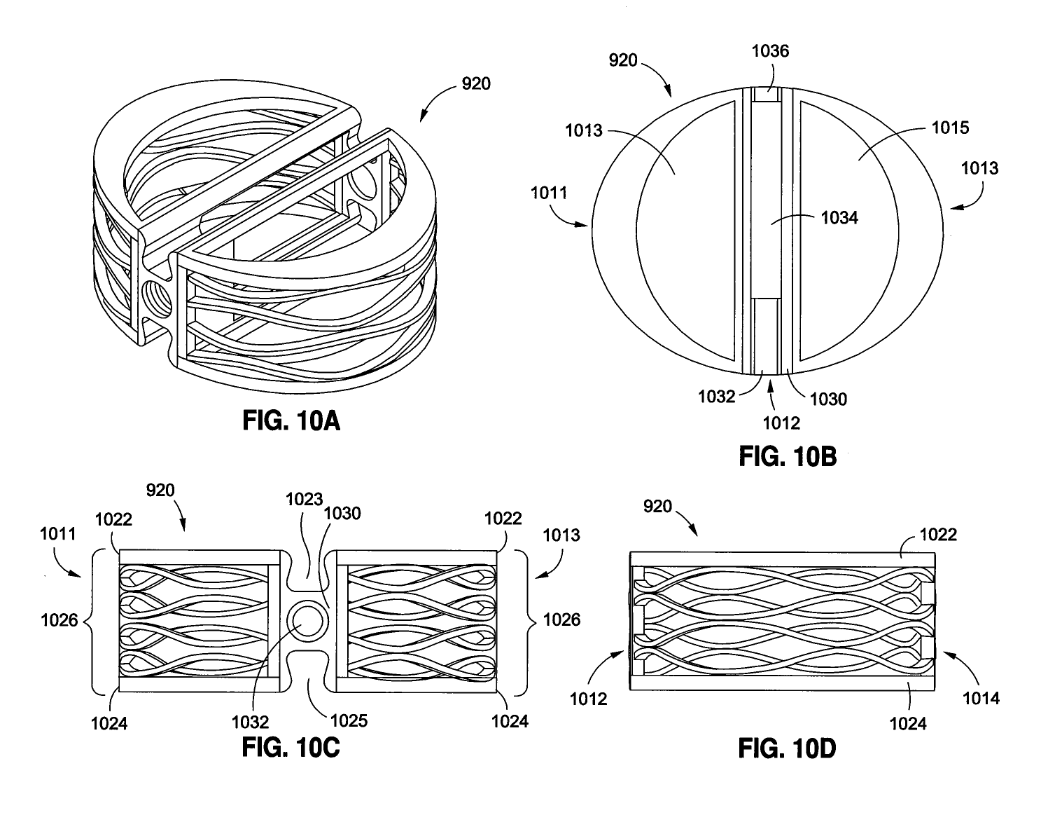

As shown in FIGS. 10A-10D, the skeletal body 920 may have a first (top) ellipsoidal frame 1022, a second (bottom) ellipsoidal frame 1024, a pressure redistribution member 1026 coupled between the first and second ellipsoidal frames 1022 and 1024. The first ellipsoidal frame 1022 may define a first alignment trench 1023 for receiving and guiding the first alignment bar 936 of the first ellipsoidal pad 930. Similarly, the second ellipsoidal frame 1024 may define a second alignment trench 1025, which may be used for receiving and guiding the second alignment bar 946 of the second ellipsoidal pad 940.

The skeletal body 920 may include a center member 1030, which may be coupled between the first and second ellipsoidal frames 1022 and 1024. The center member 1030 may define an installation channel 1032 for engaging the stopper 950. Moreover, the center member 1030 may help define the first and second fusion channels 1013 and 1015 by dividing both the first and second ellipsoidal frames 1022 and 1024 in the middle. Furthermore, the center member 1030 may have a center compartment 1034 for placing the anchoring device 960.

The pressure redistribution member 1026 may be used for implementing the shock absorbing functionality of the spinal disc. Particularly, the pressure redistribution member 1026 may balance and evenly distribute the pressure received by the first and second ellipsoidal frames 1022 and 1024. As such, the IVB spacer 900 may protect the vertebral bodies from sudden and/or uneven impact redistributing the pressure evenly across the surfaces of the vertebral bodies. The pressure redistribution member 1026 may be implemented by various structures.

In one embodiment, for example, the pressure redistribution member 1026 may be implemented by a set or stack of wave lines or structures, which may function as a contiguous pressure redistributor between the first and second ellipsoidal frames 1022 and 1024. The set of wave lines may have an up and down configuration in the shape of a wave or a curve. The stack of wave lines may be substantially rigid with little to no displacement. Alternatively, the stack of wave lines may be adjustable, replaceable, and/or removable. As such, the height of the pressure redistribution member 1026 may have an adjustable range of about 2%. Each of the wave lines may contact with one or more wave lines at one or more locations. The stack of wave lines may form a mesh cylindrical membrane surrounding a cylindrical space defined by the first and second ellipsoidal frames 1022 and 1024. Advantageously, the cylindrical membrane may help retain the bone putty within the first and second fusion channels 1013 and 1015 during the spinal fusion process. In another embodiment, for example, the pressure redistribution member 1026 may be implemented by a series of helicoidal wave lines.

Exemplary embodiments of the invention have been disclosed in an illulstrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. An inter-vertebral-body (IVB) spacer for placement between first and second vertebral bodies, the spacer comprising:
a first pad having a first contact surface configured to contact the first vertebral body;
a second pad having a second contact surface configured to contact the second vertebral body; and
a skeletal body positioned between the first and second pads, and having:
a first frame configured to secure the first pad, support the first contact surface, and arrange the first contact surface along a first plane,
a second frame configured to secure the second pad, support the second contact surface, and arrange the second contact surface along a second plane, such that the second plane forms an inter-pad angle with the first plane, and
a support member coupled between the first and second frames, and configured to maintain a distance between the first and second frames.

2. The IVB spacer of claim 1, wherein:
the skeletal body is configured to receive a bone putty for facilitating a bone fusion between the first and second vertebral bones,
the first pad defines a first opening allowing the bone putty to contact the first vertebral bone, and
the second pad defines a second opening allowing the bone putty to contact the second vertebral bone, such that the bone putty connects the first and second vertebral bones.

3. The IVB spacer of claim 1, wherein the inter-pad angle ranges from about 0 degree to about 20 degrees.

4. The IVB spacer of claim 1, wherein the first frame forms a first angle with the first plane, and the second frame forms a second angle with the second plane, such that a sum of the first and second angels is substantially the same as the inter-pad angle.

5. The IVB spacer of claim 1, wherein:
the first pad has a first alignment bar,
the first frame of the skeletal body has a first trench configured to receive and secure the first alignment bar of the first pad,
the second pad has a second alignment bar, and
the second frame of the skeletal body has a second trench configured to receive and secure the second alignment bar of the second pad.

6. The IVB spacer of claim 1, wherein:
the first pad has a plurality of first keels forming on the first contact surface, and configured to anchor the first pad to the first vertebral bone, and
the second pad has a plurality of second keels forming on the second contact surface, and configured to anchor the second pad to the second vertebral bone.

7. The IVB spacer of claim 1, wherein:
the skeletal body is made of a material selected from a group consisting of titanium, titanium alloys, titanium oxide, cobalt chrome, 316L stainless steel, nitinol, nickel alloys, ceramic, pyrolictic carbon, carbon fiber, and combinations thereof, and
the first and second pads are made of a material selected from a group consisting of ceramic, pyrolictic carbon, carbon fiber, polyether ether ketone (PEEK), ultra high molecular weight polyethylene (UHMWPE), and combinations thereof.

8. The IVB spacer of claim 1, further comprising:
an anchoring device disposed between the first and second frames of the skeletal body, and configured to protrude the first and second pads and anchor the skeletal body to the first and second vertebral bones.

9. The IVB spacer of claim 1, further comprising:
a stopper configured to be coupled to the skeletal body and prevent the first and second pads from detaching from the first and second frames.

10. The IVB spacer of claim 9, wherein the stopper has:
a first flange configured to contact the first frame and prevent the first pad from leaving the first frame, and
a second flange configured to contact the second frame and prevent the second pad from leaving the second frame.

11. The IVB spacer of claim 9, wherein:
the skeletal body has a installation channel between the first and second frames, and
the installation channel has an inner threaded surface configured to be engaged the stopper.

12. An inter-vertebral-body (IVB) spacer for placement between first and second vertebral bodies, the spacer comprising:
a first thermal plastic pad having a first contact surface configured to contact the first vertebral body;
a second thermal plastic pad having a second contact surface configured to contact the second vertebral body;
a rigid skeletal body positioned between the first and second pads, and having:
a first frame configured to secure the first pad, support the first contact surface, and arrange the first contact surface along a first plane,
a second frame configured to secure the second pad, support the second contact surface, and arrange the second contact surface along a second plane, such that the second plane forms an inter-pad angle with the first plane, and
a support member coupled between the first and second frames, and configured to maintain a distance between the first and second frames; and
a stopper configured to be coupled to the rigid skeletal body and prevent the first and second thermal plastic pads from detaching from the first and second frames.

13. The spacer of claim 12, wherein:
the rigid skeletal body is configured to receive a bone putty for facilitating a bone fusion between the first and second vertebral bones,
the first thermal plastic pad defines a first opening allowing the bone putty to contact the first vertebral bone,
the second thermal plastic pad defines a second opening allowing the bone putty to contact the second vertebral bone, such that the bone putty connects the first and second vertebral bones.

14. The spacer of claim 12, wherein the inter-pad angle ranges from about 0 degree to about 20 degrees.

15. The spacer of claim 12, wherein:
the first pad has a first alignment bar,
the first frame of the skeletal body has a first trench configured to receive and secure the first alignment bar of the first pad,
the second pad has a second alignment bar, and
the second frame of the skeletal body has a second trench configured to receive and secure the second alignment bar of the second pad.

16. The spacer of claim 12, further comprising:
an anchoring device disposed between the first and second frames of the rigid skeletal body, and configured to protrude the first and second thermal plastic pads and anchor the rigid skeletal body to the first and second vertebral bones.

17. An inter-vertebral-body (IVB) spacer for placement between first and second vertebral bodies, the spacer comprising:
a first thermal plastic pad having a first contact surface configured to contact the first vertebral body;
a second thermal plastic pad having a second contact surface configured to contact the second vertebral body;
a rigid skeletal body positioned between the first and second pads, and having:
a first frame configured to secure the first pad, support the first contact surface, and arrange the first contact surface along a first plane,
a second frame configured to secure the second pad, support the second contact surface, and arrange the second contact surface along a second plane, such that the second plane forms an inter-pad angle with the first plane, and
a support member coupled between the first and second frames, and configured to maintain a distance between the first and second frames;
a stopper configured to be coupled to the rigid skeletal body and prevent the first and second thermal plastic pads from detaching from the first and second frames; and
an anchoring device disposed between the first and second frames of the rigid skeletal body, and configured to protrude the first and second thermal plastic pads and anchor the rigid skeletal body to the first and second vertebral bones.

18. The spacer of claim 17, wherein:
the rigid skeletal body is configured to receive a bone putty for facilitating a bone fusion between the first and second vertebral bones,
the first thermal plastic pad defines a first opening allowing the bone putty to contact the first vertebral bone,
the second thermal plastic pad defines a second opening allowing the bone putty to contact the second vertebral bone, such that the bone putty connects the first and second vertebral bones.

19. The spacer of claim 17, wherein:
the first thermal plastic pad has a plurality of first keels forming on the first contact surface, and configured to anchor the first thermal plastic pad to the first vertebral bone, and
the second thermal plastic pad has a plurality of second keels forming on the second contact surface, and configured to anchor the second thermal plastic pad to the second vertebral bone.

20. The spacer of claim 17, wherein:
the skeletal body has a installation channel between the first and second frames, and
the installation channel has an inner threaded surface configured to be engaged the stopper.
